(19) United States
(12) Patent Application Publication
(10) Pub. No.: US 2026/0207751 A1
(43) Pub. Date: Jul. 23, 2026

(54) LOW-AGGREGATE OPHTHALMIC NANO-FORMULATION, PREPARATION METHOD THEREFOR, AND USE THEREOF

(71) Applicant: SHENYANG PHARMACEUTICAL UNIVERSITY, Shenyang, Liaoning (CN)

(72) Inventors: Mingxi QIAO, Shenyang, Liaoning (CN); Wenmin WANG, Shenyang, Liaoning (CN); Yan GAO, Shenyang, Liaoning (CN); Meiqi CHEN, Shenyang, Liaoning (CN); Dawei CHEN, Shenyang, Liaoning (CN)

(73) Assignee: SHENYANG PHARMACEUTICAL UNIVERSITY, Shenyang, Liaoning (CN)

(21) Appl. No.: 19/144,322

(22) PCT Filed: Nov. 3, 2023
(86) PCT No.: PCT/CN2023/129737

§ 371 Date: Jun. 27, 2025

(30) Foreign Application Priority Data
Dec. 27, 2022 (CN) 202211687184.4

(51) Int. Cl. A61K 47/44; A61K 9/51; A61K 45/00; A61K 47/14; A61K 47/24; A61K 47/28

(52) U.S. Cl. CPC A61K 47/44; A61K 9/5123; A61K 45/00; A61K 47/14; A61K 47/24; A61K 47/28

(57) ABSTRACT

The present disclosure pertains to the technical field of pharmaceutical formulations and particularly relates to a low-aggregate ophthalmic nano-formulation for efficient drug delivery, a preparation method therefor, and use thereof. The use mainly relates to the treatment of ocular diseases. The described low-aggregate ophthalmic nano-formulation of the present disclosure is prepared from a drug molecule, a phospholipid or a derivative thereof, and an amphiphilic surfactant. The concentration of the phospholipid or the derivative thereof is 0.05%-60% by weight, and the concentration of the amphiphilic surfactant is 0.05%-25% by weight. The ocular delivery system of the low-aggregate nano-formulation prepared in the present disclosure has the advantages of a particle size smaller than 10 nm, good tissue permeability, good stability, and a low irritation level, which greatly improve the bioavailability of ocular drug administration.

### CROSS-REFERENCE TO RELATED APPLICATION

This application claims the benefit of Chinese Application No. 2022116871844, filed Dec. 27, 2022. The Application No. 2022116871844 is hereby incorporated by reference in its entirety.

### TECHNICAL FIELD

The present disclosure pertains to the technical field of pharmaceutical formulations and particularly relates to a low-aggregate ophthalmic nano-formulation for efficient drug delivery, a preparation method therefor, and use thereof. The use mainly relates to the treatment of ocular diseases.

### DESCRIPTION OF RELATED ART

In recent years, with changes in lifestyle, increased work-related stress and the frequency of eye use, the incidence of eye diseases has increased year by year. Ocular topical administration is the most commonly used drug delivery method for the treatment of ocular diseases, which has the advantages of simple use and high patient acceptance. Because of the unique physiological properties of the eye, ophthalmic preparations administered in the form of eye drops are highly susceptible to being lost from the eye. The bioavailability of topical ophthalmic preparations is generally less than 5%, which greatly hinders their therapeutic effect. The main barriers to anterior segment diseases include: corneal barrier, conjunctival barrier, etc., and the main barriers to posterior segment diseases also include: vitreous barrier, scleral barrier, choroidal barrier, etc. The cornea is located in the anterior part of the eyeball, which is composed of epithelial cell layer, Bowman's membrane, stromal layer, Descemet's membrane and endothelial cell layer, and is one of the most important barriers to drug delivery in the eye. Corneal epithelial cell layer is composed of lipophilic epithelial cells, which is the barrier for hydrophilic drug molecules to penetrate. Corneal stromal layer is composed of water, collagen, proteoglycans and corneal stromal cells, which accounts for 90% of corneal thickness and is the barrier for lipophilic drug molecules to penetrate. Therefore, the penetration of eye drops to the cornea is very limited, and the permeability of the cornea is of great significance in terms of drug treatment.

With the development of nano-formulation technology, new ocular topical drug delivery systems, such as nanoemulsions, liposomes and micelles etc., have provided technical means to improve the efficiency of ocular drug delivery. Nanoemulsions and liposomes help improve the corneal penetration and ocular bioavailability of drugs, such as the O/W cyclosporin A nanoemulsion (Restasis®) developed by Allergan. Although nanoemulsions and liposomes have improved the bioavailability of topical ocular drug delivery, there are still some technical problems to be solved, for example, the particle size of nanoemulsions and liposomes is usually about 100-200 nm, and their permeability in ocular tissues needs to be further improved. Compared with liposomes and fat emulsions, micelles have a smaller particle size, generally in the range from 20 to 50 nm, and better permeability in eye tissues. For example, Cequa®, a cyclosporin A nano-micelle developed by Sun Pharma, is the first ophthalmic micelle preparation approved by the FDA. However, in order to maintain the stability of ordinary micellar particles, the aggregation number of carrier molecules and drug molecules is relatively high, and the particle size of the formed micelles is generally larger than 20 nm. Therefore, the permeability of the micelles in eye tissues still needs to be improved.

Considering the physiological characteristics of ocular tissues, nanoparticles after administration primarily permeate in the ocular tissues by diffusion, and the diffusion behavior was significantly related to the particle size of the nanoparticles. The smaller the particle size of the nanoparticles, the more conducive to their diffusion and the better the permeability in the ocular tissues. The molecular aggregation number of nanoparticles can significantly affect their particle size. The smaller the molecular aggregation number and the smaller the particle size, the more conducive to their diffusion, penetration and drug release in eye tissues. For the field of drug delivery technology, there is a huge technical challenge to prepare nanoparticles with a particle size of less than 10 nm, because 10 nm is only about the size of 30 water molecules. In general, for organic molecules with molecular weight between 300 and 1000 Da, the size of a single molecule is about 1 to 1.5 nm, and nanoparticles with a size of less than 10 nm mean that the number of molecular aggregation is quite low. If nanoparticles with a size of less than 10 nm can be designed, they will have a diffusion capacity close to that of drug molecules, and can significantly improve the permeability and bioavailability of ophthalmic administration.

However, there are no reports of low-aggregate ophthalmic nano-formulation with particle size less than 10 nm.

### SUMMARY

In order to overcome the defects in the prior art, the inventor unexpectedly found that phospholipids and their derivatives can change the self-assembly behavior of the molecules under the induction of certain amphiphilic surfactant molecules to form low-aggregate nanoparticles with a particle size of less than 10 nm.

On such a basis, the disclosure aims to provide a low-aggregate ophthalmic nano-formulation, and a preparation method and use thereof. The low-aggregate ophthalmic nano-formulation of the disclosure is prepared from a drug molecule, a phospholipid or a derivative thereof, and an amphiphilic surfactant. The ocular delivery system of the low-aggregate nano-formulation prepared by the disclosure has the advantages of a particle size of less than 10 nm, good tissue permeability, good stability and low irritation, and greatly improves the bioavailability of the ocular drug administration.

In order to achieve the above objective of the present disclosure, a technical proposal is adopted below:

In the first aspect, the disclosure provides a low-aggregate ophthalmic nano-formulation for efficient ocular delivery of a drug, the ophthalmic nano-formulation comprising a drug molecule, a phospholipid or a derivative thereof, and an amphiphilic surfactant.

Alternatively, in the ophthalmic nano-formulation above, the drug molecules participate in the self-assembly process of the carrier molecules to form a stable drug-loaded low-aggregate nano-formulation.

Preferably, the drug molecule is a hydrophobic drug molecule.

More preferably, the drug molecule is selected from one or more of immunosuppressive drugs (e.g., cyclosporin A, tacrolimus, etc.), steroid drugs (e.g., loteprednol, dexamethasone, fluorometholone, prednisone acetate, etc.), prostaglandin drugs (e.g., latanoprost, travoprost, bimatoprost, tafluprost, etc.), non-steroidal anti-inflammatory drugs (e.g., diclofenac, pranoprofen, and bromfenac, etc.), antibiotics (e.g., levofloxacin, moxifloxacin, gatifloxacin, azithromycin, etc.), antiviral drugs (e.g., acyclovir, ganciclovir, ribavirin, etc.), antifungals (e.g., natamycin, amphotericin B, etc.), antihistamines (e.g., olopatadine, azelastine, levocabastine, etc.), or atropine or rebamipide.

Alternatively, in the ophthalmic nano-formulation above, the phospholipid or the pharmaceutically acceptable derivative thereof comprises a class of phospholipid molecules or derivatives thereof widely found in organisms, which are molecules with amphiphilic chemical structures.

Furthermore, the phospholipid or the pharmaceutically acceptable derivative thereof is selected from one or more of natural phospholipids, semi-synthetic phospholipids and synthetic phospholipids. The natural phospholipid is selected from natural glycerophospholipid or natural sphingomyelin, and the natural glycerophospholipid comprises one or more of soybean lecithin, yolk lecithin, cardiolipin, phosphatidylethanolamine, phosphatidylglycerol and phosphatidylinositol. The semi-synthetic phospholipid is selected from one or more of hydrogenated soybean lecithin, hydrogenated yolk lecithin and hydrogenated sunflower lecithin. The synthetic phospholipid is selected from one or more of synthetic glycerophospholipid, synthetic sphingomyelin and polyethylene glycol phospholipid derivatives. The synthetic glycerophospholipid is selected from at least one glycerophospholipid with polar groups of choline, ethanolamine, serine, glycerol and inositol, wherein the fatty acids esterified at position sn-1 and sn-2 are linear or branched, saturated or unsaturated C4-C24 fatty acids. The glycerophospholipid with a polar group of choline may be selected from distearoyl phosphatidylcholine, dipalmitoyl phosphatidylcholine, dioleoyl phosphatidylcholine, dimyristoyl phosphatidylcholine, dilauroyl phosphatidylcholine, and dioctanoyl phosphatidylcholine; the glycerophospholipid with a polar group of glycerol may be selected from the group consisting of distearoyl phosphatidylglycerol, dipalmitoyl phosphatidylglycerol, dimyristoyl phosphatidylglycerol, dilauroyl phosphatidylglycerol, dioctanoyl phosphatidylglycerol, and salts thereof; the glycerophospholipid with a polar group of ethanolamine can be selected from distearoyl phosphatidylethanolamine, dipalmitoyl phosphatidylethanolamine, dioleoyl phosphatidylethanolamine, dimyristoyl phosphatidylethanolamine, and dilauroyl phosphatidylethanolamine; the glycerophospholipid with a polar group of inositol can be selected from one or more of distearoyl phosphatidylinositol, dipalmitoyl phosphatidylinositol, dioleoyl phosphatidylinositol, dimyristoyl phosphatidylinositol and dilauroyl phosphatidylinositol. The polyethylene glycol phospholipid derivative may be selected from dimyristoyl glycerol polyethylene glycol, distearoyl glycerol polyethylene glycol, distearoyl phosphatidylethanolamine polyethylene glycol, dimyristoyl phosphatidylethanolamine polyethylene glycol, dipalmitoyl phosphatidylethanolamine polyethylene glycol, and phosphatidylethanolamine polyethylene glycol.

Preferably, the phospholipid or pharmaceutically acceptable derivative thereof is selected from one or more of soybean lecithin, yolk lecithin, hydrogenated soybean lecithin, hydrogenated yolk lecithin, stearoylated, palmitoylated, oleoylated choline at position sn-1 and sn-2, stearoylated, palmitoylated, oleoylated glycerol at position sn-1 and sn-2, stearoylated, palmitoylated, oleoylated ethanolamine at position sn-1 and sn-2, stearoylated, palmitoylated, oleoylated inositol at position sn-1 and sn-2, dimyristoyl glycerol polyethylene glycol, distearoyl glycerol polyethylene glycol, dipalmitoyl phosphatidyl acetamide polyethylene glycol, distearoyl phosphatidyl acetamide polyethylene glycol, phosphatidylethanolamine polethylene glycol.

Phospholipids are one of the main components of biological membranes and are amphiphilic. It is well known that phospholipids and their derivative molecules can preferentially form liposomes in aqueous media. In the disclosure, it is unexpectedly found that certain amphiphilic surfactants can induce the self-assembly behavior of phospholipid molecules to form low-aggregate nanoparticles with non-liposome structure, and the most remarkable physicochemical characteristic of the low-aggregate nanoparticles is that the particle size is less than 10 nm. In addition, surfactant molecules can also interact with biological membranes or reduce the efflux of drug molecules caused by P-glycoprotein, effectively promote the penetration of drug molecules in eye tissues, and increase the bioavailability of drugs.

Alternatively, in the above ophthalmic nano-formulation, the amphiphilic surfactant is selected from one or more of polyoxyethylene surfactants and steroid surfactants. The polyoxyethylene surfactant is preferably a polyoxyethylene castor oil surfactant or a polyoxyethylene 15 hydroxystearate (Kolliphor® HS 15), and more preferably a polyoxyethylene 15 hydroxystearate (Kolliphor® HS 15). The steroid surfactant is preferably selected from the group consisting of cholesteric acid and salts thereof, cholesteric acid-amino acid conjugates and salts thereof, and more preferably deoxycholic acid, chenodeoxycholic acid salts, cholesteric acid-glycine conjugates and salts thereof, and cholesteric acid-taurine conjugates and salts thereof. Even more preferably, the steroid surfactant is glycocholic acid and salts thereof, deoxycholic acid and salts thereof, glycodeoxycholic acid and salts thereof, taurocholic acid and salts thereof, and taurodeoxycholic acid and salts thereof.

Alternatively, the above ophthalmic nano-formulation also comprises other common adjuvants used in the ophthalmic formulation.

Other common adjuvants for the ophthalmic formulation include, but not limited to, buffers, lubricants, osmo-regulators, antibacterial agents, antioxidants, bioadhesive agents, thickeners, wetting agents, and preservatives.

The ophthalmic nano-formulation is preferably formulated at pH 4-8, and the pH of the formulation can be adjusted by the addition of buffer salts. Such buffers include, but not limited to, phosphates, borates, acetates, citrates, carbonates, and mixtures thereof.

The osmo-regulators include, but not limited to, mannitol, glucose, sodium chloride, glycerol, propylene glycol, xylitol, and mixtures thereof. These osmo-regulators can be used to adjust the osmolarity of the ophthalmic nano-formulation of the present disclosure.

The preservatives include, but not limited to, benzyl alcohol, benzalkonium bromide, benzalkonium chloride, chlorhexidine, parabens such as methylparaben, ethylparaben, propylparaben, benzoic acid, and mixtures thereof. It should be noted that the preservative does not necessarily need to be added, and if the ophthalmic nano-formulation of the present disclosure is packaged in the form of a single dose, the formulation may not contain the preservative.

The low-aggregate ophthalmic nano-formulation for efficient ocular delivery of a drug also comprises one or more pharmaceutically acceptable bioadhesive agents for improving the viscosity of the formulation and further prolonging the retention time of the present formulation in the eyes. The bioadhesive agent of the present disclosure includes carboxyl polymers, such as carbomer, polycarbophil, etc.; cellulose derivatives including alkyl and hydroxyalkyl celluloses, such as methyl cellulose, hydroxypropyl cellulose, hydroxypropyl methyl cellulose, carboxymethyl cellulose, etc.; gums, such as xanthan gum, carrageenan, etc.; and other pharmaceutically acceptable polymers, including but not limited to, polyvinyl alcohol, polyvinyl pyrrolidone, poloxamer and derivatives thereof, hyaluronic acid and salts thereof, alginic acid and salts thereof, and the like. Such polymers may be used alone or in combination.

Alternatively, in the above ophthalmic nano-formulation, the ophthalmic nano-formulation has a particle size of less than 10 nm. Preferably, the particle size of the ophthalmic nano-formulation ranges from 1 nm to 10 nm. The encapsulation rate of the ophthalmic nano-formulation is greater than 90%.

Alternatively, in the above ophthalmic nano-formulation, the concentration of the phospholipid or the derivative thereof is 0.05%-60%, preferably 1% -50%, and more preferably 2%-40% by weight; the concentration of the amphiphilic surfactant is 0.05% to 25%, preferably 0.1% to 20%, and more preferably 0.5% to 15%.

In the second aspect, the present disclosure provides a method for preparing the ophthalmic nano-formulation according to the first aspect above, comprising the following steps: dissolving or dispersing the pharmaceutical component and the amphiphilic carrier in an appropriate amount of an organic solvent or a mixed solution of organic solvents, removing the solvent by evaporation under reduced pressure, mixing the residue with a solution containing water-soluble adjuvants to homogeneity, filtering the solution with a microporous membrane, and sterilizing to obtain the solution of the drug-loaded ophthalmic nano-formulation. The prepared solution of the drug-loaded ophthalmic nano-formulation is filled into a multi-dose or single-dose packaging container through aseptic aliquoting.

Preferably, the organic solvent can be selected from one or more of: methanol, ethanol, isopropanol, dichloromethane, or chloroform.

In addition, those skilled in the art will know that the ophthalmic nano-formulation described in the first aspect above can also be prepared by other methods known in the field of pharmaceutical formulation.

In the third aspect, the present disclosure provides the use of the ophthalmic nano-formulation described in the first aspect above in the preparation of an ophthalmic drug.

Alternatively, in the above use, the ophthalmic drug is used in the prevention, treatment or adjuvant therapy of ocular diseases.

Preferably, the ophthalmic drug has higher drug permeability and bioavailability.

As compared with the prior art, the disclosure has the following beneficial effects:

The mechanism of the present low-aggregate ophthalmic nano-formulation for efficient ocular delivery of the drug is as follows: the drug, the phospholipid and the derivative thereof, and the amphiphilic surfactant are self-assembled to form low-aggregate nanoparticles for the delivery of active drug molecules. It has been surprisingly found that the pharmaceutical active ingredient can form particles with a particle size ranging from 1 nm to 10 nm with the phospholipid and the derivative thereof and the amphiphilic surfactant, which is much smaller than that of common amphiphilic polymeric micelles and the commercially available Cequa® product. The most remarkable advantage of the present low-aggregate ophthalmic nano-formulation for efficient ocular delivery of the drug is the good permeability in the ocular tissues. As the size of the low-aggregate ophthalmic nano-formulation is close to the size of common drug molecules, the phospholipid and the derivative thereof have good cell affinity with the components of the cell membrane. Moreover, the amphiphilic surfactant can promote the penetration behavior of the drug components in the ocular tissues, and the penetration of the drugs in the ocular tissues is far stronger than that in other common nano-delivery systems.

### BRIEF DESCRIPTION OF DRAWINGS

FIG. 1 is a representative result of the particle size measured for Sample 3 of Example 1 according to the present disclosure.

FIG. 2 is an in vitro release profile of the low-aggregate nanoparticles from Example 3 of the present disclosure.

FIG. 3 shows the result of the corneal staining by fluorescein in Example 4 according to the present disclosure.

FIG. 4 shows the tear film break-up time of Example 4 of the present disclosure.

FIG. 5 shows the changes in the concentrations of the inflammatory factors in Example 4 of the present disclosure.

FIG. 6 shows the distribution profile within the ocular tissue and plasma according to Example 5 of the present disclosure.

### DETAILED DESCRIPTION

The disclosure is further described below with reference to specific Examples. It should be understood that the specific Examples described herein are merely illustrative of the disclosure, and are not intended to limit the scope of the disclosure.

If no specific technology or conditions are specified in the Examples, the technology or conditions described in the literatures in this field or in the product specification shall be followed. If no manufacturer is indicated for the reagent or instrument used, it is a conventional product that can be purchased through regular channels.

The experimental methods in the following Examples are all conventional methods, unless otherwise specified. The test materials used in the following Examples are commercially available, unless otherwise specified.

### Example 1: Preparation and Pharmaceutical Properties of the Drug-Loaded Low-Aggregate Nanoparticles

The required phospholipid or its derivative, amphiphilic surfactant and active drug were weighed out according to the proportion in Table 1, into which about 10 mL of ethanol solution or other organic solution was added. The mixture was stirred until a clear solution was obtained. The solvent was then evaporated by heating under vacuum, into which water or buffer salt solution was added, mixed and stirred to homogeneity to obtain the solution of the drug-loaded low-aggregate nanoparticles.

TABLE 1
Formulation composition of the active drug-loaded low-aggregate nanoparticles

| Sample | Phospholipid or its | | | Concentration |
|---|---|---|---|---|
| name | derivatives (%) | Surfactant (%) | Active drug | (%) |
| 1 | Lecithin (2.5%) | Polyoxyethylene- | Latanoprost | 0.005 |
| | | 15-hydroxystearate | | |
| | | (1%) | | |
| 2 | Soybean phospholipid | Polyoxyethylene 40 | Tafluprost | 0.0015 |
| | (5%) | hydrogenated castor | | |
| | | oil (2%) | | |
| 3 | Soybean phospholipid | Sodium | Cyclosporin A | 0.09 |
| | (15%) | glycocholate (5%) | | |
| 4 | Phosphatidylethanolamine | Sodium | Dexamethasone | 0.1 |
| | (28%) | glycodeoxycholate | | |
| | | (7%) | | |
| 5 | Phosphatidylglycerol | Sodium | Loteprednol | 0.5 |
| | (35%) | taurodeoxycholate | | |
| | | (15%) | | |
| 6 | Polethylene glycol | Sodium | Diclofenac | 0.1 |
| | phosphatidylethanolamine | deoxycholate (18%) | | |
| | (45%) | | | |
| 7 | Dipalmitoyl | Potassium | Cyclosporin A | 0.09 |
| | phosphatidylcholine (55%) | glycocholate (20%) | | |
| 8 | Hydrogenated soybean | Sodium | Tacrolimus | 0.1 |
| | lecithin (60%) | taurocholate (25%) | | |

The preparation method in Comparative Example 1 was identical to that in Example 1.

TABLE 2
Formulation composition of the nanoparticles
of Comparative Example 1

| | | | | Concen- |
|---|---|---|---|---|
| Sample | | | Active | tration |
| name | Surfactant | | drug | (%) |
| 9 (Commercial | Polyoxyethylene 40 | Octylphenol | Cyclo- | 0.09% |
| available | hydrogenated | polyether | sporin | |
| Cequa ® | castor oil | | A | |
| product) | | | | |

TABLE 3
Main pharmaceutical properties of Sample 1-9

| Sample | Encapsulation rate | Particle size | |
|---|---|---|---|
| name | (%) | (nm) | Distribution |
| 1 | 98.5 | 5.73 | 0.175 |
| 2 | 99.2 | 6.76 | 0.189 |
| 3 | 95.7 | 5.94 | 0.161 |
| 4 | 98.9 | 4.89 | 0.205 |
| 5 | 99.1 | 5.87 | 0.213 |
| 6 | 98.6 | 7.71 | 0.202 |
| 7 | 98.8 | 6.37 | 0.210 |
| 8 | 99.0 | 5.82 | 0.237 |
| 9 | 95.2 | 20.32 | 0.206 |

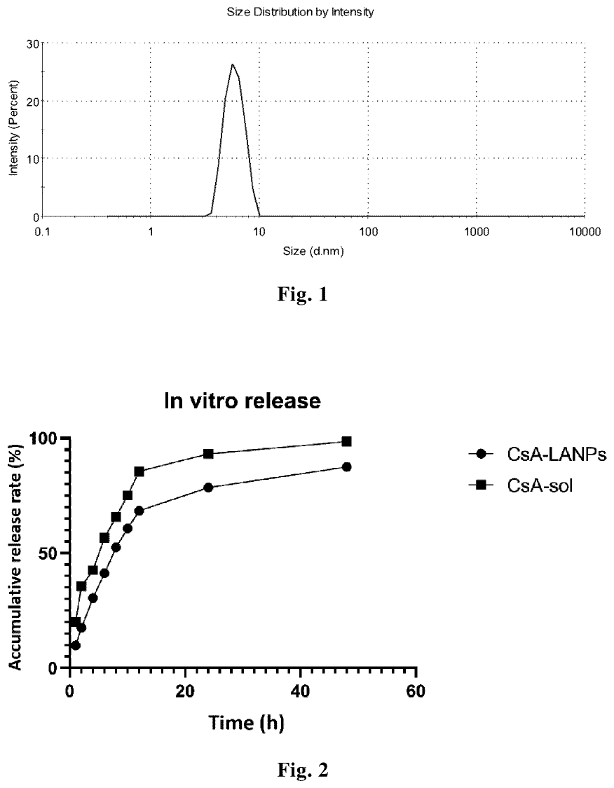

FIG. 1 is a representative result of the particle size measured for Sample 3 of Example 1. The results in Table 3 show that the average particle size by optical intensity of the low-aggregate nanoparticles prepared by the disclosure is less than 10 nm, which is much smaller than that of the commercially available Cequa® product (20.32 nm).

### Example 2. Low-Aggregate Nanoparticle Formulation with Water-Soluble Adjuvants

The required phospholipid or its derivative, amphiphilic surfactant and active drug were weighed out according to the proportion in Table 4, into which about 10 mL of ethanol solution or other organic solution was added. The mixture was stirred until a clear solution was obtained. The solvent was then evaporated by heating under vacuum, into which the solution of water-soluble adjuvants was added, mixed and stirred to homogeneity. The pH was adjusted to about 6.8 with NaOH or HCl, and the osmotic pressure was adjusted to 280-300 mOSmol/kg by the addition of NaCl. A constant volume of 100 mL was then obtained by adding deionized water. The solution was sterilized by filtration or autoclaving, and then filled to give the final solution.

TABLE 4
Formulation composition of low-aggregate nanoparticles

| | | | |
|---|---|---|---|
| Ingredient | 1 | 2 | 3 |

| | | |
|---|---|---|
| Cyclosporin A | 0.09 | g |
| Sodium glycocholate | 5 | g |
| Soybean phospholipid | 15 | g |

| | | |
|---|---|---|
| Tafluprost | 0.0015 | g |
| Soybean phospholipid | 5 | g |
| Polyoxyethylene 40 | 2 | g |

| hydrogenated castor oil | | |
|---|---|---|
| Loteprednol | 0.5 | g |
| Phosphatidylglycerol | 35 | g |
| Sodium taurodeoxycholate | 15 | g |

TABLE 5
Composition of the water-soluble adjuvant solution

| Ingredient | 1 | | 2 | | 2 | | 2 | | 3 | |
|---|---|---|---|---|---|---|---|---|---|---|
| Disodium | 0.81 | g | 0.81 | g | 0.81 | g | 0.81 | g | 0.81 | g |
| hydrogen | | | | | | | | | | |
| phosphate | | | | | | | | | | |
| Monosodium | 0.93 | g | 0.93 | g | 0.93 | g | 0.93 | g | 0.93 | g |
| phosphate | | | | | | | | | | |
| Benzalkonium | 0.02 | g | 0.02 | g | N/A | | N/A | | N/A | |
| chloride | | | | | | | | | | |
| PVP-K30 | 1.8 | g | | | | | | | | |
| PVP-K90 | | | 1.2 | g | | | | | | |
| HPC | | | | | 0.2 | g | | | | |
| HPMC | | | | | | | 0.5 | g | | |
| Hyaluronic acid | | | | | | | | | 0.1 | |
| NaCl | Q.S. | | Q.S. | | Q.S. | | Q.S. | | Q.S. | |
| Added to | 100 | mL | 100 | mL | 100 | mL | 100 | mL | 100 | mL |

### Example 3: In Vitro Release of Cyclosporin A-Loaded Low-Aggregate Nanoparticles

2 mL of Sample 3 solution in Example 1 was added to a dialysis bag (MW=3500), which was then tightly sealed at both ends and placed into 10 mL of PBS buffer solution (pH=7.4) containing 1% SDS (w/w) in a rotary shaker, shaken at 37° C. and 100 rpm. After 1, 2, 4, 6, 8, 10, 24 and 48 h, 1 mL of the release medium was taken out and supplemented with another 1 mL of release medium. After the sample was filtered through a 0.22 μm filter, it was diluted appropriately with methanol. The drug content in the medium was determined by HPLC and the cumulative release rate was calculated.

The results in FIG. 2 show that the in vitro release rate of the low-aggregate nanoparticles is slightly lower than that of the drug solution, showing sustained release characteristics to some extent.

### Example 4: Pharmacokinetic Study of Cyclosporine A-Loaded Low-Aggregate Nanoparticles

0.2% benzalkonium chloride solution was instilled into both eyes of healthy Japanese white rabbits once each in the morning, noon and evening for 7 consecutive days. Tear film break-up time (TBUT) and tear secretion volume were monitored throughout the process of modeling and treatment. The TBUT was less than 10 s and the tear secretion volume was less than 5 mm, demonstrating that the dry eye model was successfully established. The rabbits in each group of the dry eye model were treated with normal saline, the solution of Comparative Example 1 (Cequa), and the solution of Sample 3 of Example 1 for 2 weeks.

(1) Corneal Staining with Sodium Fluorescein

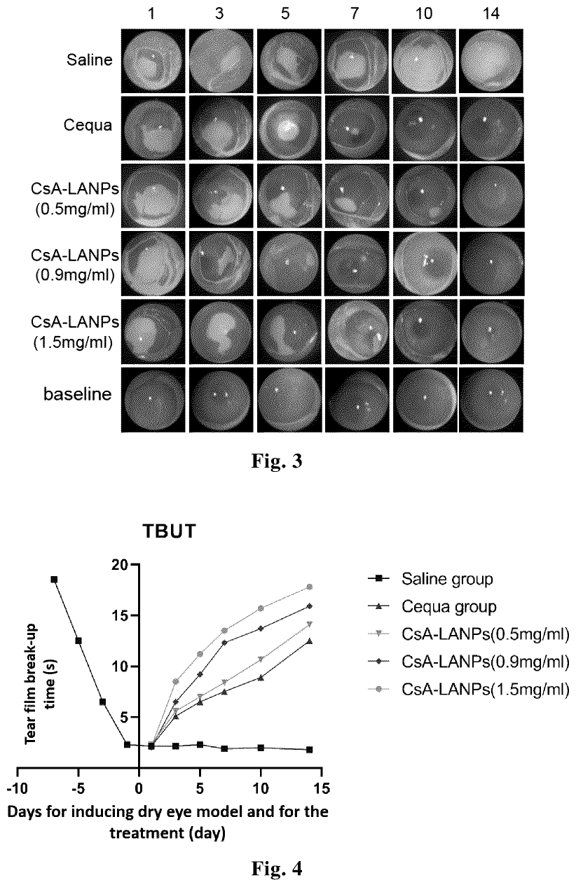

As shown in FIG. 3, the corneal staining results with sodium fluorescein showed that the fluorescent staining in the saline group was not improved and became more serious after 14 days of treatment. Compared with the saline group, the staining with sodium fluorescein in the formulation groups was significantly improved. The degree of improvement for corneal staining was significantly better in the Sample 3 group than in the Comparative Example 1 group.

### (2) Tear Film Break-Up Time

As shown in FIG. 4, the TBUT in the saline group was substantially unchanged, while it was significantly improved in the formulation groups. The TBUT values for the Sample 3 and the solution of Comparative Example 1 were both greater than 10 s. The TBUT value for the Sample 3 group was significantly greater than the Comparative Example 1 group.

### (3) Tear Inflammatory Factors

After treatment with different eye drops, the change trend of the inflammatory factor concentration in tears was determined. Tears were collected using a capillary at the indicated time points, and the concentrations of IL-1, IL-2, IL-6, and TNF-α were measured.

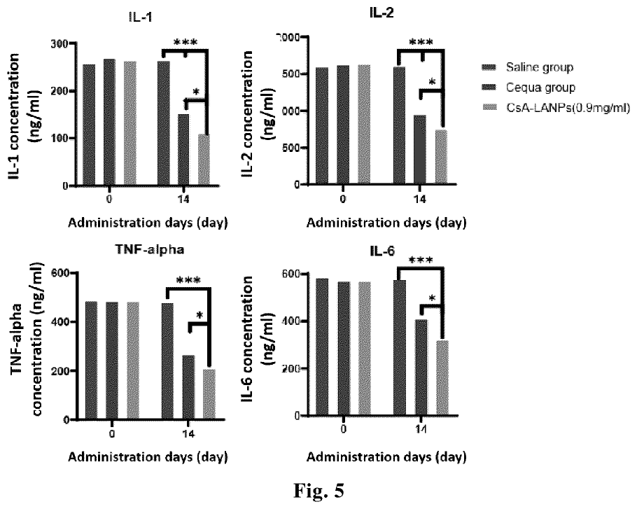

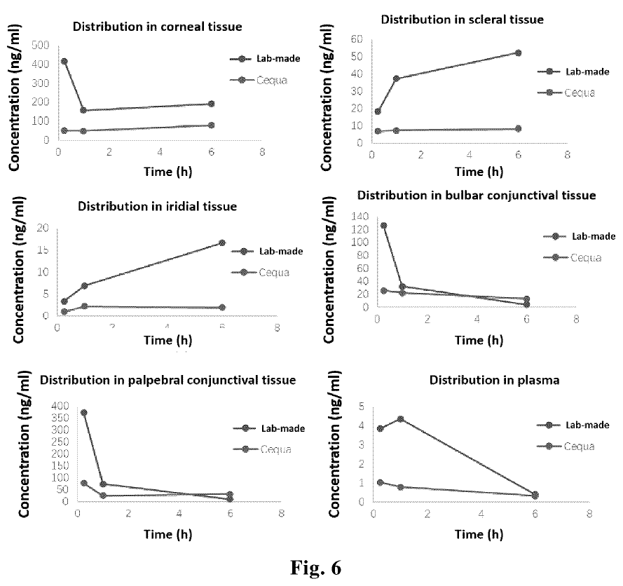

As shown in FIG. 5, the concentration of each inflammatory factor increased to a very high level before treatment, which then significantly decreased after 14 days of treatment. The levels of inflammatory factors in the Sample 3 group and the Comparative Example 1 group, and the Sample 3 group and the blank control group were statistically analyzed by independent sample T test, and the results showed that the levels of inflammatory factors in the Sample 3 group were significantly lower than that in the Comparative Example 1 (Cequa) group (p<0.05), and in the blank control group (p<0.001).

### Example 5: Distribution in Ocular Tissue and Pharmacokinetics of Cyclosporin A-Loaded Low-Aggregate Nanoparticles

The Japanese white rabbits were randomly divided into two groups, which were then unilaterally administered with the Sample 3 solution of Example 1 (0.09%) and the solution of Comparative Example 1 (Cequa) (0.09%) at a single dose of 50 μL/eye. Blood samples were collected at 0. 25 h, 1 h and 6 h after administration, and the animals were sacrificed under anesthesia, and the bulbar conjunctiva, palpebral conjunctiva, cornea, sclera and iris tissues were collected. 0.1 g of each tissue sample was accurately weighed out, into which an appropriate amount of 50% methanol solution was added. The mixture was then homogenized with a tissue homogenizer (8000 rpm for 3 times) to give a 5% tissue homogenate. Subsequently, the homogenate was placed into a 2 mL polypropylene tube, and stored at −60° C. to −90° C. The concentration of the drug was determined by LC/MS/MS after the above treatment. The distribution in ocular tissue and the pharmacokinetic profile for the lab-made formulation (Sample 3 of Example 1) and the control formulation (Comparative Example 1) are shown in FIG. 5. It can be seen from the results that the distribution concentration of the cyclosporin A-loaded low-aggregate nanoparticles of the present disclosure in the main ocular tissues and the concentration in the plasma are significantly higher than those of the commercially available control formulation. The above results have demonstrated that the low-aggregate nanoparticles of the present disclosure has better ocular tissue permeability and absorption behavior and high bioavailability for ocular administration compared to the commercially available formulation of Comparative Example 1.

It will be apparent to those skilled in the art that various modifications and variations can be made in the present disclosure without departing from the spirit and scope of the disclosure. Thus, the present disclosure is intended to include such modifications and variations provided they come within the scope of the appended claims and their equivalents.

1. A low-aggregate ophthalmic nano-formulation, wherein:
the ophthalmic nano-formulation comprises a drug molecule, a phospholipid or derivative thereof, and an amphiphilic surfactant, wherein the particle size of the ophthalmic nano-formulation is less than 10 nm, and the encapsulation rate of the ophthalmic nano-formulation is greater than 90%.

2. The ophthalmic nano-formulation according to claim 1, wherein:
the drug molecule is a hydrophobic drug molecule, and the drug molecule is selected from one or more of the following: immunosuppressive drugs, steroid drugs, prostaglandin drugs, non-steroidal anti-inflammatory drugs, antibiotics, antiviral drugs, antifungal drugs, antihistamine drugs, or atropine or rebamipide, and the particle size of the ophthalmic nano-formulation ranges from 1 nm to 10 nm.

3. The ophthalmic nano-formulation according to claim 1, wherein:
the phospholipid or its derivative is selected from one or more of the following: natural phospholipids, semi-synthetic phospholipids, or synthetic phospholipids; and the amphiphilic surfactant is selected from one or more of steroid surfactants or polyoxyethylene surfactants.

4. The ophthalmic nano-formulation according to claim 3, wherein:
the natural phospholipid is selected from one or more of the following: soybean lecithin, yolk lecithin, cardiolipin, phosphatidylethanolamine, phosphatidylglycerol, sphingomyelin, and phosphatidylinositol; the semi-synthetic phospholipid is selected from one or more of the following: hydrogenated soybean lecithin, and hydrogenated yolk lecithin; the synthetic phospholipid is selected from one or more of the following: glycerophospholipids, synthetic sphingomyelins and polyethylene glycol phospholipid derivatives, wherein their polar group is choline, ethanolamine, serine, glycerol or inositol, and the position sn-1 and sn-2 are substituted by linear or branched, saturated or unsaturated C4-C24 fatty acid; the steroid surfactant is selected from one or more of: cholesteric acid and salts thereof, cholesteric acid-amino acid conjugates and salts thereof; the polyoxyethylene surfactant is selected from one or more of polyoxyethylene castor oil surfactant and polyoxyethylene 15 hydroxystearate (Kolliphor® HS 15).

5. The ophthalmic nano-formulation according to claim 1, wherein:
the concentration of the phospholipid or the derivative thereof is 0.05-60%, preferably 1-50% by weight percentage; and the concentration of the amphiphilic surfactant is 0.05% -25%, preferably 0.1%-20%.

6. The ophthalmic nano-formulation according to claim 1, wherein:
the ophthalmic nano-formulation also comprises other common adjuvants used in ophthalmic formulations, wherein other commonly used adjuvants of the ophthalmic formulation are selected from one or more of the following: buffers, lubricants, osmo-regulators, antibacterial agents, antioxidants, bioadhesive agents, thickeners, wetting agents or preservatives, and the pH of the ophthalmic nano-formulation is 4-8, preferably, the pH of the ophthalmic nano-formulation is 6-7.5.

7. The ophthalmic nano-formulation according to claim 6, wherein:
the buffer is selected from one or more of: phosphate, borate, acetate, citrate, or carbonate; the osmo-regulator is selected from one or more of: mannitol, glucose, sodium chloride, glycerol, propylene glycol, or xylitol; the preservative is selected from one or more of: benzyl alcohol, benzalkonium bromide, benzalkonium chloride, chlorhexidine, parabens such as methylparaben, ethylparaben, propylparaben, or benzoic acid; and the bioadhesive agent is selected from one or more of: carboxyl polymers, cellulose derivatives, gums, polyvinyl alcohols, polyvinyl pyrrolidone, poloxamer or derivatives thereof, hyaluronic acid or salts thereof, or alginic acid or salts thereof.

8. The method for preparing the ophthalmic nano-formulation according to claim 1, wherein: the preparation method comprises the following steps:
S1: dissolving or dispersing the drug molecules and the amphiphilic carrier in an appropriate amount of an organic solvent or a mixed solution of organic solvents, removing the solvent by evaporation under reduced pressure, mixing the residue with a solution containing water-soluble adjuvants to homogeneity, filtering the solution with a microporous membrane, and sterilizing to obtain the solution of the drug-loaded ophthalmic nano-formulation;
S2: filling the prepared drug-loaded ophthalmic nano-formulation solution into a multi-dose or single-dose packaging container by aseptic aliquoting.

9. Use of the ophthalmic nano-formulation according to claim 1, in the preparation of an ophthalmic drug.

10. The use according to claim 9, wherein:
the ophthalmic drug is used for the prevention, treatment or adjuvant therapy of ocular diseases, and the ophthalmic drug has enhanced drug permeability and bioavailability.

11. Use of the ophthalmic nano-formulation according to claim 8 in the preparation of an ophthalmic drug.

12. The use according to claim 11, wherein:
the ophthalmic drug is used for the prevention, treatment or adjuvant therapy of ocular diseases, and the ophthalmic drug has enhanced drug permeability and bioavailability.
